(19) United States
(12) Patent Application Publication
(10) Pub. No.: US 2021/0052557 A1
(43) Pub. Date: Feb. 25, 2021

(54) COMPOSITIONS FOR TREATING INFECTIVE ARTERIAL DISEASES AND RELATED CONDITIONS

(71) Applicant: CENTRE FOR DIGESTIVE DISEASES, Five Dock (AU)

(72) Inventor: Thomas Julius Borody, Five Dock (AU)

(21) Appl. No.: 16/958,299

(22) PCT Filed: Feb. 1, 2019
(86) PCT No.: PCT/AU2019/050078

§ 371 Date: Jun. 26, 2020

Related U.S. Application Data
(60) Provisional application No. 62/625,259, filed on Feb. 1, 2018.

(51) Int. Cl. A61K 31/438; A61K 31/65; A61K 31/7052; A61K 31/7048; A61K 31/4178; A61K 31/355; A61K 31/593; A61K 31/198; A61K 31/675; A61K 31/52; A61K 31/573; A61K 31/519; A61K 31/454; A61K 31/429; A61K 38/13; A61K 31/436; A61P 31/04; A61P 9/10; A61K 45/06; A61K 38/19; A61K 38/21; A61K 31/343

(52) U.S. Cl. CPC A61K 31/438; A61K 31/343; A61K 31/7052; A61K 31/7048; A61K 31/4178; A61K 31/355; A61K 31/593; A61K 31/198; A61K 31/675; A61K 31/52; A61K 31/573; A61K 31/519; A61K 31/454; A61K 31/429; A61K 38/13; A61K 31/436; A61P 31/04; A61P 9/10; A61K 45/06; A61K 38/191; A61K 38/212; A61K 38/215; A61K 38/217; A61K 31/65

(57) ABSTRACT

In alternative embodiments, provided are pharmaceutical compositions and methods for treatment, amelioration and prevention of infection-associated blood vessel diseases, and also for the treatment, amelioration and prevention of non-vessel diseases affected by infective agents which can be treated by these compositions. In alternative embodiments, one common pathogen targeted by compositions and methods as provided herein is Chlamydia and Chlamydophila species, including pneumoniae, trachomatis and psittaci species which infect humans, including Chlamydophila penumoniae which also infects humans. In alternative embodiments, pathogens targeted and infections (diseases) treated ameliorated, or prevented by compositions and methods as provided herein include Mycoplasma, Listeria, Leptospirosis, Q fever or Coxiella burnetii infection, Lyme disease or Lyme borreliosis or any Borrelia infection, and Bartonella or of the family Bartonellaceae, including cat scratch disease.

### FIELD

This invention generally relates to medicine and infectious disease. In alternative embodiments, provided are pharmaceutical compositions and methods for treatment, amelioration and prevention of infection-associated blood vessel diseases, and also for the treatment, amelioration and prevention of non-vessel diseases affected by infective agents which can be treated by these compositions. In alternative embodiments, one common pathogen targeted by compositions and methods as provided herein is Chlamydia and Chlamydophila species, including pneumoniae, trachomatis and psittaci species which infect humans, including Chlamydophila penumoniae which also infects humans. In alternative embodiments, pathogens targeted and infections (diseases) treated ameliorated, or prevented by compositions and methods as provided herein include Mycoplasma, Listeria, Leptospirosis, Q fever or Coxiella burnetii infection, Lyme disease or Lyme borreliosis or any Borrelia infection, and Bartonella or of the family Bartonellaceae, including cat scratch disease.

### BACKGROUND

A typical infections as those mentioned above, infect various regions of the body and are often carried in circulating white cells such as monocytes/macrophages. They generally infect intracellular locations, for example, intima layer cells of arteries, causing chronic inflammation in arteries followed by edema, necrosis, foam cell formation which results in plaque formation. Vascular disease such as coronary heart disease and stroke remain the major cause of morbidity and mortality worldwide. Particularly in China the escalating numbers of patients with coronary and peripheral artery disease has been described as being epidemic proportions.

Both coronary heart disease (CAD) and peripheral vascular disease are currently treated by identifying and targeting ‘risk factors’ since these are the only parameters to treat in the absence of an underlying cause of these conditions.

The development of atheroma in the vessel walls after invasion by macrophages carrying Chlamydophila pneumoniae (Cpn) to the intima begins with visible foam cells, followed by edema or swelling, then destruction of muscle cells and necrosis with fully formed plaque projecting into the lumen of the pulsating epicardial arteries. Complications of plaque formation can follow such as partial plaque rupture and local activation of the clotting cascade which may occlude the blood vessel partially or completely. This may lead to death of distant tissues/muscle which is called ‘myocardial infarction’—and may be accompanied by severe pain, and arrythmias. When this happens in the brain it is called ‘ischemic stroke’. A similar process may also take place in peripheral vessels such as leg and foot vessels, causing them to be cold, painful, lose sensation or ultimately necrosis of toes.

Conventional therapy for vascular disease aims to open the vessel lumen and to prevent or reverse clot formation with reduction of plaque formation. Hence, the treatments and preventative therapies target risk factors such as hypertension, smoking and dyslipidaemia.

Treatment of Cpn targets an infective bacterium which can be visualised in and cultured from arterial tissue. Chlamydophila pneumoniae a name interchangeable with Chlamydia pneumonia, has only been recently described, and is known as a microorganism which can infect numerous tissues. It is responsible for up to one third of community acquired pneumonia, bronchitis, chronic obstructive airways disease and asthma, and has now been recognised to be present in atherosclerotic plaque. Hence, Cpn has been incriminated in causality of the inflammation in the lungs and arteries and may well be the cause of the known arterial inflammation that predates plaque development and so causes vascular disease such as coronary heart disease. It has also been shown that Cpn Chlamydophila pneumoniae is capable of using cholesterol, and in its metabolic cycles Cpn is capable of producing cells containing lipids and so it causes foam cells to appear at the site of the formation of atherosclerotic plaque. This explains better the relationship of cholesterol and fats in the blood vessel and the action of Cpn, and explains why arterial wall lipid deposition is related to food-derived cholesterol and triglycerides. Cpn is an obligate intracellular pathogen that grows within macrophages and in vessel wall cells including muscle cells. Infection with Cpn is characterised by intracellular persistence following the infections and it has been estimated that about 50% of the population are sero-positive for Cpn in adult life and most persons acquire the infection via the respiratory route generally in teenage years. Related Chlamydia infections such as Chlamydia trachomatis can cause acute infections such as conjunctivitis, sexually transmitted diseases, as well as chronic infection that can lead to trachoma, tubal infection and infertility, pelvic inflammatory disease, and reactive arthritis. For Cpn, acute infections are localised in the airways with pneumonia and bronchitis being the most common condition. The chronic sequelae developing from acute or from asymptomatic infections cannot be at times definitively attributed to Cpn. Although, evidence is accumulating that it may lead to debilitating asthma and even fatal heart disease conditions. It may also cause reactive arthritis, possibly late onset Alzheimer's disease, multiple sclerosis and complications of these diseases.

Cpn like other members of the genus has the characteristic biphasic lifecycle between the infectious, metabolically inert elementary body (EB) and the non-infectious metabolically active reticulate body (RD). EB's are internalised into the pathogen modified phagosomes which avoid destruction with lysosomes. This is referred to as an inclusion and this ability of Cpn to enter non-productive growth state is often termed persistence or dormancy. Not all persons infected with Cpn develop vascular disease or asthma, however recovery rates of the microorganisms have ranged between 20% and 60% of sites of atherosclerotic tissue and up to 100% when serial histology sections are taken. The organism has not been recovered from normal vascular tissue. Animal models have been developed, in which infection with Cpn is followed in an accelerated fashion by development of atherosclerotic plaque.

A number of therapies have been trialed, but generally speaking, only monotherapy has been used i.e. a single antibiotic was given to patients. Azithromycin has been used and showed some benefit, but this was not sustained. Benefit was also seen with use of roxithromycin. Tetracyclines have been used as monotherapy but without clinical benefit and since then it has been discovered that the bacteriostatic tetracyclines may convert the metabolically-active reticulate bodies (RB) into a metabolically-inactive elementary body (EB), which is metabolically inert and less affected by antibiotics.

The use of single antibiotics in treatment of Cpn which at best is difficult to eradicate, due to is dormant forms, has the potential of causing serious adverse consequence in large populations of the world and that many will develop resistant infections for which there is really unlikely to be any effective therapies. In many cases suppression rather than eradication occurs and following the withdrawal of the antibiotics the suppressed pathogen can become reactivated and develops illness with a resistant strain.

Persistent chlamydial infections can be established in vitro using some cytokines, antibiotics and by deprivation of certain nutrients. This may also be occurring in vivo. When growth inhibitory factors are removed aberrant bodies which are reticulate bodies (RBs) in atypical form can be restored to normal. The characteristic of persistent Cpn infection is the development of large RB forms along with the absence of EB's. Hence, in a cell environment in which a condition of nutritional stress is created the Cpn organisms fall into a ‘persistent state’. The presence of a persistent state causes consistent presentation to that individual's immune system and could lead to potentially deleterious immune affects such as chronic arthritis and inflammation in arteries. Repeated infection can also cause a similar affect as persistent infection. Removal of various antibiotics especially Penicillins, Ciprofloxacin or Ofloxacin can induce the persistence infection to be significantly decreased and allow the persistent Chlamydia to turn into infectious EB's which are more easily treatable and opens the door to better eradication. Cyclic starting and stopping some such antibiotics in a timely fashion improves recruitment into treatable state.

There is little teaching of the use of combined antibiotics. Yet with use of in vitro sensitivity testing there is no relationship to in vivo clinical result and such an approach should no longer be used. Sensitivities should still be tested but in chronic longstanding infection a minimum of three antimicrobial agents should be combined to try and prevent widespread resistance developing. Hence, there is a need for better methods of treating conditions associated with Cpn and other infections mentioned above. There is also a need to treat the initial infection effectively with a combination of antibiotics to prevent it going into a chronic phase with consequences of ongoing disease and heightened bacteria resistance.

### SUMMARY OF INVENTION

In alternative embodiments, provided are pharmaceutical compositions, formulations, or products of manufacture, comprising at least three different antibiotics selected from the group consisting of:

(a)
(i) a macrolide (e.g., azithromycin, clarithromycin, erythromycin, fidaxomicin, telithromycin), a quinolone (e.g., fluoroquinolone), chloramphenicol (e.g., PENTAMYCETIN™, CHLOROMYCETIN™), a cephalosporin, a nitazoxanide (e.g., ALINIA™, NIZONIDE™), a furazolidone (e.g., FUROXONE™, DEPENDAL-M™), a lincomycin or clindamycin (e.g., CLEOCIN™, CLINACIN™), an aminoglycoside (e.g., streptomycin), a carbapenem (e.g., imipenem, meropenem, ertapenem, doripenem, panipenem or betamipron, biapenem, tebipenem), a glycopeptide (e.g., vancomycin, teicoplanin, telavancin, ramoplanin, decaplanin), a glycocycline (e.g., tigecycline), a streptogramin (e.g., quinupristin/dalfopristin, pristinamycin, virginiamycin), a rifamycin (e.g., rifampicin (or rifampin), rifabutin, rifapentine, rifalazil, rifaximin, rifamycin SV (or AEMCOLO™)) an ansamycin (e.g., streptovaricin, geldanamycin, rifamycin), a sulphonamide (e.g., sulfadimethoxine, sulfamethoxypyridazine, sulfametoxydiazine, sulfadoxine, sulfametopyrazine, terephtyl), an oxazolidinone (e.g., 2-oxazolidone, linezolid, posizolid, tedizolid, radezolid, cycloserine) and/or a nitroimidazole (e.g., metronidazole, tinidazole, nimorazole, dimetridazole, pretomanid, ornidazole, megazol, azanidazole); or
(ii) rifalazil, rifabutin (e.g., MYCOBUTIN™), omiganan (or omiganan pentahydrochloride), radezolid (RX-1741), torezolid, RWJ-416457, ceftobiprole (or ZEVTERA™, MABELIO™), a nitrofuran (e.g., difurazone, furazolidone, nifurfoline, nifuroxazide, nifurquinazol), isoniazid (e.g., HYDRA™, HYZYD™, ISOVIT™), ceftaroline (e.g., ceftaroline fosamil, or TEFLARO™, ZINFORO™), iclaprim, garenoxacin, cethromycin (e.g., RESTANZA™) and/or telithromycin (e.g., KETEK™) (part of the macrolide group), doxycycline, tigecycline (e.g., TYGACIL™), minocycline (e.g., MINOCIN™, AKAMIN™) and/or a tetracycline (e.g., SUMYCIN™); or,
(iii) a combination of (i) and (ii);
   (b) the pharmaceutical composition, formulation, or a product of manufacture of (a), further comprising at least four antibiotics, and optionally a metronidazole (e.g., FLAGYL™, METRO™), tinidazole (e.g., FASIGYN™, SIMPLOTAN™, TINDAMAX™), ornidazole (e.g., XYNOR™), secnidazole (e.g., FLAGENTYL, SINDOSE™, SECNI™L, SOLOSEC™) roxithromycin, doxycycline (e.g., DORYX™, DOXYHEXAL™, DOXYLIN™), minocycline (e.g., MINOCIN™, MINOMYCIN™, AKAMIN™), clarithromycin (e.g., BIAXIN™) and/or nitroimidazole (e.g., metronidazole, tinidazole, nimorazole, dimetridazole, pretomanid, ornidazole, megazol, azanidazole) is the at least fourth antibiotic,
   and optionally the nitroimidazole is formulated for administration to be cycled 2 weeks on and 2 weeks off.

In alternative embodiments, the at least three different antibiotics comprises:

(a) rifampicin, azithromycin and moxifloxacin, optionally in a ramping-up dose (or formulated for a ramping up dosage regimen);
(b) at least comprises rifabutin, and clarithromycin or azithromycin;
(c) at least comprises rifabutin and minocycline;
(d) at least comprises rifabutin and azithromycin;
(e) at least comprises rifabutin and clarithromycin; or
(f) comprises rifabutin, azithromycin and minocycline.

In alternative embodiments, provided is a pharmaceutical composition, formulation, or a product of manufacture, comprising:
(a) at least three different antibiotics selected from the group consisting of:
(i) a macrolide (e.g., azithromycin, clarithromycin, erythromycin, fidaxomicin, telithromycin), a quinolone (e.g., fluoroquinolone), chloramphenicol (e.g., PENTAMYCETIN™, CHLOROMYCETIN™), a cephalosporin, a nitazoxanide (e.g., ALINIA™, NIZONIDE™), a furazolidone (e.g., FUROXONE™, DEPENDAL-M™), a lincomycin or clindamycin (e.g., CLEOCIN™, CLINACIN™), an aminoglycoside (e.g., streptomycin), a carbapenem (e.g., imipenem, meropenem, ertapenem, doripenem, panipenem or betamipron, biapenem, tebipenem), a glycopeptide (e.g., vancomycin, teicoplanin, telavancin, ramoplanin, decaplanin), a glycocycline (e.g., tigecycline), a streptogramin (e.g., quinupristin/dalfopristin, pristinamycin, virginiamycin), a rifamycin (e.g., rifampicin (or rifampin), rifabutin, rifapentine, rifalazil, rifaximin, rifamycin SV (or AEMCOLO™)) an ansamycin (e.g., streptovaricin, geldanamycin, rifamycin), a sulphonamide (e.g., sulfadimethoxine, sulfamethoxypyridazine, sulfametoxydiazine, sulfadoxine, sulfametopyrazine, terephtyl), an oxazolidinone (e.g., 2-oxazolidone, linezolid, posizolid, tedizolid, radezolid, cycloserine) and/or a nitroimidazole (e.g., metronidazole, tinidazole, nimorazole, dimetridazole, pretomanid, ornidazole, megazol, azanidazole); or
(ii) rifalazil, rifabutin (e.g., MYCOBUTIN™), omiganan (or omiganan pentahydrochloride), radezolid (RX-1741), torezolid, RWJ-416457, ceftobiprole (or ZEVTERA™, MABELIO™), a nitrofuran (e.g., difurazone, furazolidone, nifurfoline, nifuroxazide, nifurquinazol), isoniazid (e.g., HYDRA™, HYZYD™, ISOVIT™), ceftaroline (e.g., ceftaroline fosamil, or TEFLARO™, ZINFORO™), iclaprim, garenoxacin, cethromycin (e.g., RESTANZA™) and/or telithromycin (e.g., KETEK™) (part of the macrolide group), doxycycline, tigecycline (e.g., TYGACIL™), minocycline (e.g., MINOCIN™, AKAMIN™) and/or a tetracycline (e.g., SUMYCIN™); or,
(iii) a combination of (i) and (ii); or
(b) the pharmaceutical composition, formulation, or a product of manufacture of (a), further comprising at least a fourth antibiotic, and optionally a metronidazole (e.g., FLAGYL™, METRO™), tinidazole (e.g., FASIGYN™, SIMPLOTAN™, TINDAMAX™), ornidazole (e.g., XYNOR™), secnidazole (e.g., FLAGENTYL, SINDOSE™, SECNI™L, SOLOSEC™) roxithromycin, doxycycline (e.g., DORYX™, DOXYHEXAL™, DOXYLIN™), minocycline (e.g., MINOCIN™, MINOMYCIN™, AKAMIN™), clarithromycin (e.g., BIAXIN™) and/or nitroimidazole (e.g., metronidazole, tinidazole, nimorazole, dimetridazole, pretomanid, ornidazole, megazol, azanidazole) is the at least fourth antibiotic,
and optionally the nitroimidazole is formulated for administration to be cycled 2 weeks on and 2 weeks off.

In alternative embodiments, the at least three different antibiotics comprise clarithromycin, rifabutin and furazolidone. Alternatively, the at least three antibiotics comprise rifabutin, azithromycin and doxycycline. The rifabutin, azithromycin and doxycycline may be combined with vitamin D.

In alternative embodiments, the pharmaceutical compositions, formulations, or products of manufacture further comprises: (a) a vitamin E, a tocotrienol, a natural tocopherol or a tocochromanol, a vitamin D, or any combination thereof, wherein optionally the vitamin D is formulated for use in doses of up to about 5000 to 20,000 units per day, optionally to achieve blood levels of about 150 to 375 nmol/l; (b) a penicillamine, or DEPEN™ or CUPRIMINE™; (c) an acetylcysteine or N-acetylcysteine (NAC), or ACETADOTE™, FLUIMUCIL™, MUCOMYST™; or (d) any combination of (a) to (c). In alternative embodiments, Vitamin D needs to be used in higher than expected doses, as we have shown in patients, for example, at about 5000 to 20,000 units per day to achieve blood levels near the top of normal range of around 200 to 375 nmol/l. These levels are non-toxic, for toxicity to occur much higher levels need to be reached.

In alternative embodiments, the pharmaceutical compositions, formulations, or products of manufacture further comprises: an agent selected from other medications used in the management of coronary and other vascular disease, other medications that enhance host defence mechanisms important in the eradication of intracellular pathogens, selective and non-selective cyclooxygenase inhibitors; other antiplatelet drugs; betablockers; antiarrhythmics; calcium channel blockers; other anticoagulant drugs; nitrate medicines and HMG-Coareductase inhibitors; immune response modifiers selected from cytokines; colony stimulating factors; tumour necrosis factors alpha and beta; interferon alpha, beta and gamma; peptides which bind to macrophage and lymphocyte surface receptors: glycoproteins which mimic cytokines; and other mediator molecules; prednisone and related steroids, azathioprine, mofetil mycofenolate and related purine antagonists, cyclophosphamide and related alkylating agents, methotrexate and related folate antagonists, thalidomide, chloroquine and related antimalarial compounds, levamisole, cyclosporin A, rapamycin and/or FK506.

In alternative embodiments, the pharmaceutical compositions, formulations, or products of manufacture are formulated for parenteral or enteral delivery, or for oral delivery, optionally in a capsule, a tablet, a geltab or a solution or a liquid, or as an aerosol, wherein optionally the at least three different antibiotics, or at least two antibiotic, or all active agents are in one formulation, optionally a capsule, a tablet, a geltab or a solution or liquid.

In alternative embodiments, the product of manufacture is an implant.

In alternative embodiments, provided are methods for inhibiting development of atheroma in arterial vessel walls after invasion by macrophages carrying Chlamydophila pneumoniae (Cpn) to arterial intima, or for the treatment, amelioration and prevention of:
an infection-associated blood vessel disease,
a non-vessel disease affected by an infective agent,
a disease or condition caused by Chlamydia or a Chlamydophila species, including pneumoniae, trachomatis and psittaci species which infect humans, including Chlamydophila pneumoniae,
a disease or condition caused by Mycoplasma, Listeria, Leptospirosis, Q fever or Coxiella burnetii infection, Lyme disease or Lyme borreliosis or any Borrelia infection, and Bartonella or of the family Bartonellaceae, including cat scratch disease,
arterial inflammation, atheroma or arterial foam cells,
chronic arthritis, or
Alzheimer's disease or a dementia, wherein optionally the dementia is vascular dementia, Lewy body dementia or frontotemporal dementia, or a dementia caused or a result of normal pressure hydrocephalus, Parkinson's disease dementia, syphilis or Creutzfeldt-Jakob disease, the method comprising administration to an individual in need thereof a pharmaceutical composition, a formulation, or a product of manufacture as provided herein.

In alternative embodiments, the methods further comprise administration of:

(a) vitamin E or tocotrienol or equivalents (optionally comprising vitamin E or tocochromanol either as a natural Tocopherol or Tocotrienol), optionally added in a cyclic fashion, optionally from between daily to weekly administrations; and/or
(b) vitamin D, optionally administered to upper limit of normal levels, optionally administered in dosages of at least about 5,000 to about 20,000 units per day, optionally until levels are reached which are also capable of killing an intracellular infectious agent and reducing intracellular persistence of the intracellular infectious agent, optionally Chlamydophila pneumonia within macrophages.

In alternative embodiments, of the methods the pharmaceutical composition or formulation is administered parenterally or enterally, or orally, optionally in a capsule, a tablet, a geltab or a solution or a liquid, or as an aerosol, wherein optionally the at least three different antibiotics, or at least two antibiotic, or all active agents are in one formulation, optionally a capsule, a tablet, a geltab or a solution or liquid, and optionally each active agent is formulated in a separate product of manufacture, optionally each active agent in a separate capsule, tablet, geltab, solution or liquid.

In alternative embodiments, provided are uses of a pharmaceutical composition, formulation, or a product of manufacture as provided herein for inhibiting development of atheroma in arterial vessel walls after invasion by macrophages carrying Chlamydophila pneumoniae (Cpn) to arterial intima, or for the treatment, amelioration and prevention of:
an infection-associated blood vessel disease,
a non-vessel disease affected by an infective agent,
a disease or condition caused by Chlamydia or a Chlamydophila species, including pneumoniae, trachomatis and psittaci species which infect humans, including Chlamydophila pneumoniae,
a disease or condition caused by Mycoplasma, Listeria, Leptospirosis, Q fever or Coxiella burnetii infection, Lyme disease or Lyme borreliosis or any Borrelia infection, and Bartonella or of the family Bartonellaceae, including cat scratch disease,
arterial inflammation, atheroma or arterial foam cells,
chronic arthritis, or
Alzheimer's disease or a dementia, wherein optionally the dementia is vascular dementia, Lewy body dementia or frontotemporal dementia, or a dementia caused or a result of normal pressure hydrocephalus, Parkinson's disease dementia, syphilis or Creutzfeldt-Jakob disease.

In alternative embodiments, provided are pharmaceutical compositions, formulations, products of manufacture as provided herein for inhibiting development of atheroma in arterial vessel walls after invasion by macrophages carrying Chlamydophila pneumoniae (Cpn) to arterial intima, or for use in the treatment, amelioration and prevention of:
an infection-associated blood vessel disease,
a non-vessel disease affected by an infective agent,
a disease or condition caused by Chlamydia or a Chlamydophila species, including pneumoniae, trachomatis and psittaci species which infect humans, including Chlamydophila pneumoniae,
a disease or condition caused by Mycoplasma, Listeria, Leptospirosis, Q fever or Coxiella burnetii infection, Lyme disease or Lyme borreliosis or any Borrelia infection,, and Bartonella or of the family Bartonellaceae, including cat scratch disease,
arterial inflammation, atheroma or arterial foam cells,
chronic arthritis, or
Alzheimer's disease or a dementia, wherein optionally the dementia is vascular dementia, Lewy body dementia or frontotemporal dementia, or a dementia caused or a result of normal pressure hydrocephalus, Parkinson's disease dementia, syphilis or Creutzfeldt-Jakob disease.

The details of one or more exemplary embodiments of the invention are set forth in this description. Other features, objects, and advantages of the invention will be apparent from the description and from the claims.

All publications, patents, patent applications cited herein are hereby expressly incorporated by reference for all purposes.

In alternative embodiments, provided is the use of the pharmaceutical composition, formulation, or product of manufacture of any one of claims 1 to 5 for the manufacture of a medicament for inhibiting development of atheroma in arterial vessel walls after invasion by macrophages carrying Chlamydophila pneumoniae (Cpn) to arterial intima, or for use in the treatment, amelioration and prevention of:
an infection-associated blood vessel disease,
a non-vessel disease affected by an infective agent,
a disease or condition caused by Chlamydia or a Chlamydophila species, including pneumoniae, trachomatis and psittaci species which infect humans, including Chlamydophila pneumoniae,
a disease or condition caused by Mycoplasma, Listeria, Leptospirosis, Q fever or Coxiella burnetii infection, Lyme disease or Lyme borreliosis or any Borrelia infection, and Bartonella or of the family Bartonellaceae, including cat scratch disease,
arterial inflammation, atheroma or arterial foam cells,
chronic arthritis, or
Alzheimer's disease or a dementia, wherein optionally the dementia is vascular dementia, Lewy body dementia or frontotemporal dementia, or a dementia caused or a result of normal pressure hydrocephalus, Parkinson's disease dementia, syphilis or Creutzfeldt-Jakob disease.

### DETAILED DESCRIPTION

In alternative embodiments, provided are pharmaceutical compositions and methods for treatment, amelioration and prevention of infection-associated blood vessel diseases, and also for the treatment, amelioration and prevention of non-vessel diseases affected by infective agents which can be treated by these compositions. In alternative embodiments, one common pathogen targeted by compositions and methods as provided herein is Chlamydia and Chlamydophila species, including pneumoniae, trachomatis and psittaci species which infect humans, including Chlamydophila pneumoniae which also infects humans. In alternative embodiments, pathogens targeted and infections (diseases) treated, ameliorated, or prevented by compositions and methods as provided herein include Mycoplasma, Listeria, Leptospirosis, Q fever or Coxiella burnetii infection, Lyme disease or Lyme borreliosis or any Borrelia infection,, and Bartonella or of the family Bartonellaceae, including cat scratch disease.

In alternative embodiments, provided herein are methods and pharmaceutical compositions for the treatment, amelioration and prevention of vascular complications of Chlamydia infections but also disorders resulting from or aggravated by such infections. Such disorders include asthma, chronic obstructive lung disease, dementia, urinary and gynaecologic mucosal Chlamydial infections.

In alternative embodiments, provided are methods for the treatment, amelioration or prevention of a condition or disease associated with infection by Chlamydial infections, including Chlamydophila pneumoniae (Cpn), or other infections as listed above, in a patient in need of such treatment or prevention.

In alternative embodiments, compositions and methods as provided herein comprise or comprise administration to an individual (e.g., a patient or an animal) of an affective amount of at least three different antibiotics selected from the group consisting of: macrolides, quinolones, chloramphenicol, cephalosporins, nitazoxanide, furazolidones, lincomycins, aminoglycosides, carbapenems, glycopeptides, glycocyclines, isoniazid, streptogramins, rifamycins/ansamycins, sulphonamides, oxazolidinones, nitrofurans and nitroimidazoles.

alternative embodiments, compositions and methods as provided herein comprise or comprise administration to an individual (e.g., a patient or an animal) of an affective amount of at least three different antibiotics selected from the group consisting of: clarithromycin, rifabutin and furazolidone. Alternatively, the at least three antibiotics comprise rifabutin, azithromycin and doxycycline. The rifabutin, azithromycin and doxycycline may be combined with vitamin D.

In alternative embodiments, compositions and methods as provided herein comprise or comprise administration to an individual (e.g., a patient or an animal) of an affective amount of at least three different antibiotics selected from the group consisting of: rifalazil, rifabutin, omiganan, radezolid (RX-1741), torezolid, RWJ-416457, ceftobiprole, ceftaroline, iclaprim, garenoxacin, cethromycin and/or telithromycin (part of the macrolide group), tigecycline, minocycline and tetracycline.

Tigecycline is a novel glycylcycline, but it remains a tetracycline derivative which has good activity against Chlamydophila pneumoniae (Cpn). A new class of macrolide derivatives called ketolides, which includes telithromycin tend to have similar high activity as clarithromycin possesses, but some isolates are more sensitive than others. The most active of the macrolides used in compositions and methods as provided herein is cethromycin. In alternative embodiments, other anti-Cpn and other infective agent medications used in compositions and methods as provided herein include vancomycin, gentamicin, rifaximin, ampicillin, streptomycin, trimethoprim/sulfamethoxazole. In alternative embodiments, novel inhibitors of peptide deformylase that can be used include NVP-PDF386/ABT/773 and Des/Quinolone/BMS/284756. These are highly powerful new antimicrobial agents emerging in the treatment of Cpn.

In alternative embodiments, a three-drug regimen as provided herein comprises rifampicin, azithromycin and moxifloxacin, optionally in a ramping-up dose, a feature observed by the inventor to permit treatment with the combination without adverse effects. In alternative embodiments, a three-drug regimen as provided herein comprises rifabutin and clarithromycin.

In alternative embodiments, provided are methods for treating, ameliorating or preventing in individuals, e.g., patients or animals, with a previous or current infection with Cpn, or other infections mentioned above by administering at least three antimicrobial agents listed above.

In alternative embodiments, to prevent the development of the persistent forms of Cpn a supplemental vitamin E or tocotrienol will be added in a cyclic fashion from between second daily to weekly; and these can comprise vitamin E or tocochromanol either as being a natural tocopherol or tocotrienol. The addition of the vitamin E derivatives can markedly reduce the aberrant body development accelerating the destruction of Chlamydia in human lymphocytes.

In alternative embodiments, compositions and methods also comprise the addition of vitamin D to upper limit of normal levels by ingesting at least 5,000-20,000 units per day, until levels are reached, which are also capable of killing the Cpn and reducing intracellular persistence of the intracellular agent.

In alternative embodiments, the cycling of nitroimidazoles such as metronidazole, tinidazole, ornidazole and secnidazole, also prevents the formation of the resistant intracellular reticulate bodies (RBs) and can be inserted to the cycling protocol of three antibiotics as the fourth antibiotic, or metronidazole, tinidazole, ornidazole, secnidazole, roxithromycin, doxycycline, minocycline, clarithromycin or nitroimidazole is the fourth antibiotic can be the fourth antibiotic, in a pharmaceutical composition, formulation, or a product of manufacture as provided herein.

In alternative embodiments, a three drug regime as provided herein comprises rifampicin, azithromycin and moxifloxacin, optionally avoiding tetracyclines as they can inhibit the production of elementary bodies.

In alternative embodiments, compositions and methods as provided herein are used for Alzheimer's disease or a dementia, wherein optionally the dementia is vascular dementia, Lewy body dementia or frontotemporal dementia, or a dementia caused or a result of normal pressure hydrocephalus, Parkinson's disease dementia, syphilis or Creutzfeldt-Jakob disease; and the compositions and methods can comprise use of: nitroimidazole, roxithromycin, doxycycline, minocycline, clarithromycin, or minocycline and clarithromycin, optionally combined with a vitamin E and/or vitamin D, optionally in a cycling fashion, or optionally including a nitroimidazole one out of two weeks, or two weeks on and two weeks off, to permit the formation of the susceptible Chlamydophila pneumoniae (Cpn) bodies.

A number of embodiments of the invention have been described. Nevertheless, it can be understood that various modifications may be made without departing from the spirit and scope of the invention. Accordingly, other embodiments are within the scope of the following claims.

### EXAMPLES

### Example 1

A 50-year-old male patient presenting with 70% left anterior descending (LAD) artery obstruction with positive stress test and reduced blood flow on sestamibi scan had failed standard cardiac treatment. He commenced treatment with three antibiotics (clarithromycin, rifabutin and furazolidone). After three months of therapy, the patient reported a complete resolution of angina pain and showed marked improvement of blood flow on sestamibi test at 6 months.

### Example 2

A 63-year-old female with a positive sestamibi test and decreased blood flow to distal epicardial arteries commenced treatment with three different antibiotics and the addition of 10,000 units vitamin D. The patient reported marked clinical improvement and the sestamibi scan was also improved at 3 months of combination rifabutin, azithromycin and doxycycline plus vitamin D.

1. A method for inhibiting development of atheroma in arterial vessel walls after invasion by macrophages carrying Chlamydophila pneumoniae (Cpn) to arterial intima, or for the treatment or prevention of an infection-associated blood vessel disease,
the method comprising administration to an individual in need thereof a pharmaceutical composition comprising at least three different antibiotics selected from:
(i) a macrolide antibiotic,
(ii) rifabutin, and
(iii) a tetracycline antibiotic or a nitrofuran antibiotic.

2. The method of claim 1, wherein the pharmaceutical composition comprises:
(a) rifabutin and minocycline;
(b) rifabutin and azithromycin;
(c) rifabutin and clarithromycin; or
(d) rifabutin, azithromycin and minocycline.

3. The method of claim 1, wherein the pharmaceutical composition comprises clarithromycin, rifabutin and furazolidone.

3. The method of claim 1, wherein the pharmaceutical composition comprises rifabutin, azithromycin and doxycycline.

5. The method of claim 1, wherein the pharmaceutical composition further comprises:
(a) vitamin E, a tocotrienol, a natural tocopherol or a tocochromanol, vitamin D, or any combination thereof,
wherein optionally the vitamin D is formulated for use in doses of up to about 5000 to 20,000 units per day, optionally to achieve blood levels of about 150 to 375 nmol/l;
(b) penicillamine;
(c) acetylcysteine or N-acetylcysteine; or
(d) any combination of (a) to (c).

6. The method of claim 1, wherein the pharmaceutical composition further comprises:
an agent selected from other medications used in the management of coronary and other vascular disease,
a medication that enhances a host defence mechanism for the eradication of an intracellular pathogen,
a selective or a non-selective cyclooxygenase inhibitor;
an antiplatelet drug;
a betablocker;
an antiarrhythmic;
a calcium channel blocker;
an anticoagulant drug;
a nitrate medicines or a HMG-Coa reductase inhibitors;
an immune response modifier, optionally selected from the group consisting of cytokines; colony stimulating factors; and tumour necrosis factors alpha and beta; interferon alpha, beta and gamma;
a peptide which bind to a macrophage or a lymphocyte surface receptor:
a glycoprotein which mimics a cytokine;
a steroid, optionally prednisone,
an azathioprine,
a purine antagonist, optionally mofetil mycofenolate,
an alkylating agent, optionally cyclophosphamide,
a folate antagonist, optionally methotrexate,
thalidomide,
an antimalarial compound, optionally chloroquine,
levamisole,
cyclosporin A,
rapamycin, and
FK506.

7. The method claim 1, further comprising administration of:
(a) vitamin E or tocotrienol or equivalents, optionally administered in a cyclic fashion, optionally from between daily to weekly administrations; and/or
(b) vitamin D,
optionally administered to upper limit of normal levels,
optionally administered in dosages of at least about 5,000 to about 20,000 units per day, optionally until levels are reached which are also capable of killing an intracellular infectious agent and reducing intracellular persistence of the intracellular infectious agent, optionally Chlamydophila pneumoniae.

8. The method claim 1, wherein the pharmaceutical composition is administered parenterally or enterally, or orally, optionally in a capsule, a tablet, a geltab or a solution or a liquid, or as an aerosol,
wherein optionally the at least three different antibiotics, or at least two antibiotics, or all active agents are in one formulation, optionally a capsule, a tablet, a geltab or a solution or liquid,
and optionally each active agent is formulated in a separate product of manufacture, optionally each active agent in a separate capsule, tablet, geltab, solution or liquid.

9. A pharmaceutical composition comprising at least three different antibiotics selected from:
(i) a macrolide antibiotic,
(ii) rifabutin, and
(iii) a tetracycline antibiotic or a nitrofuran antibiotic.

10. A pharmaceutical composition consisting essentially of three different antibiotics selected from:
(i) a macrolide antibiotic,
(ii) rifabutin, and
(iii) a tetracycline antibiotic or a nitrofuran antibiotic.

11. The pharmaceutical composition of claim 9, wherein the pharmaceutical composition comprises rifabutin and minocycline.

12. The pharmaceutical composition of claim 9, wherein the pharmaceutical composition comprises rifabutin and azithromycin.

13. The pharmaceutical composition of claim 9, wherein the pharmaceutical composition comprises rifabutin and clarithromycin.

14. The pharmaceutical composition of claim 9, wherein the pharmaceutical composition comprises rifabutin, azithromycin and minocycline.

15. The pharmaceutical composition of claim 9, wherein the pharmaceutical composition comprises clarithromycin, rifabutin and furazolidone.

16. The pharmaceutical composition of claim 9, wherein the pharmaceutical composition comprises rifabutin, azithromycin and doxycycline.

17. The pharmaceutical composition of claim 9, wherein the pharmaceutical composition is formulated for administration parenterally or enterally, or orally, optionally in a capsule, a tablet, a geltab or a solution or a liquid, or as an aerosol.
